(19) United States
(12) Patent Application Publication
(10) Pub. No.: US 2022/0110900 A1
(43) Pub. Date: Apr. 14, 2022

(54) SOLID NUTRIENT COMPOSITIONS AND ASSOCIATED METHODS

(71) Applicant: Nutrient Density Technology, LLC, Saratoga Springs, UT (US)

(72) Inventor: Joshua Martin, Saratoga Springs, UT (US)

(21) Appl. No.: 17/450,414

(22) Filed: Oct. 8, 2021

Related U.S. Application Data
(60) Provisional application No. 63/090,156, filed on Oct. 9, 2020.

(51) Int. Cl. A61K 31/198; A61K 47/12; A61K 47/26; A61K 47/46; A61K 47/02; A61K 31/522; A61K 9/00; A61K 31/145; A61K 31/155

(52) U.S. Cl. CPC A61K 31/198; A61K 47/12; A61K 47/26; A61K 47/46; A61K 31/155; A61K 31/522; A61K 9/0056; A61K 31/145; A61K 47/02

(57) ABSTRACT

Disclosed herein are oral formulations with a decreased excipient profile, comprising compositions in powdered or solid oral dosage form comprised of an active ingredient combined with an excipient blend comprising a binder and a lubricant. Further disclosed herein are methods of making oral formulations with a decreased excipient profile, comprising adding an amount of an active ingredient to an amount of an excipient blend comprising a binder and a lubricant to produce a powdered or solid oral dosage form composition. Further disclosed herein are methods of increasing absorption of one or more active ingredients in oral formulations with a decreased excipient profile, the method comprising: receiving the intraorally absorbable compositions comprising the one or more active ingredients; inserting the composition into the mouth of a subject; and allowing the composition to fully dissolve in the mouth of the subject.

### CROSS-REFERENCE TO RELATED APPLICATION

This application claims priority benefit of U.S. Provisional Patent Application Ser. No. 63/090,156 filed Oct. 9, 2020, the entire contents of which are incorporated by reference herein.

### FIELD OF INVENTION

The field of the disclosure relates generally to solid nutrient delivery products and compositions and methods for use in producing the same.

### BACKGROUND

Current nutrient delivery systems and methods can suffer from a lack of convenience, consistency, and effectiveness with respect to their use and the quantity of nutrient delivered. For example, powders and liquid reconstituted powders can be ineffective because of inconsistent and inaccurate dosing, unsustainable nutrient delivery, limited digestion and absorption capabilities, and exposure to hepatic first-pass degradation and gastric emptying processes. Powders can also be inconvenient because of messiness and spills associated with scooping, shaking and mixing that can contribute to waste; dependence on liquid and liquid containers for reconstitution; tendency to clump together; bulky storage containers; airborne powder release resulting in contamination and waste; frequent urination due to excess liquid consumption required to achieve limited absorption and systemic circulation of active ingredients; and frequent and/or intense excrement passage due to stomach irritation resulting from overdosing of active ingredients and/or digestive irritation due to bypassing important, initial digestive steps.

Alternatives to powders and liquid reconstituted powders also can suffer from limitations. For example, gummies can be extremely limited in potential deliverable active ingredient profiles and amounts, contain high levels of added sugar and/or sugar alcohols and total carbohydrates, require extremely high overall dosing and serving size (e.g. 8 gummies per serving) to achieve the relevant dose of active ingredients, and have limited product scalability due to their manufacturing process and manufacturing cost. Gels can be limited in potential deliverable active ingredient profiles, contain high levels of added sugar, and require excess filler substances. Swallowable tablets, capsules and gel caps can suffer from difficulty in swallowing, active ingredients being degraded in the gut and exposed to hepatic first-pass degradation and gastric emptying processes, each of which can limit absorption and systemic circulation of active ingredients. Gel caps can also be limited by product scalability issues due to their manufacturing process and manufacturing cost. Chewable tablets can be limited in potential deliverable active ingredient profiles; require extremely high excipient profiles; and require high amounts of added sugars, sugar derivatives, sugar substitutes, or sugar alcohol binders and fillers. Orally disintegrating tablets (ODTs) and rapid melt tablets can be extremely limited in potential deliverable active ingredient profiles; include sugars, sugar derivatives, sugar substitutes, and sugar alcohols; and have limited product availability due to manufacturing limitations. Effervescent tablets can be extremely limited in potential deliverable active ingredient profiles and amounts of active ingredient deliverable, require relatively high quantities of excipients and reactionary materials, are dependent on liquids for delivery, and have limited and expensive manufacturing and packaging requirements due to instability of reactionary ingredients. And, bottled & canned drinks (i.e. sports drinks, energy drinks) require drinking large amounts of overdosed beverages to achieve limited absorption and systemic circulation of active ingredients.

Additionally, single and multi-ingredient solid nutrient delivery formulations and products existing in the market have limited amount of flavored active ingredient profile because of the large amount of included excipients, which include sugars, sugar derivatives, sugar substitutes, and sugar alcohols that are part of the production and manufacturing process. Some examples of excipient profiles in commercially available solid dose products include (presented as percentage by weight): Nuun (Sport, Rest, Immunity, Vitamin), averages 6.75% active ingredients and 93.25% inactive ingredients and sugars; Airborne, averages 17.5% active ingredients and 82.5% inactive ingredients and sugars; Alka Seltzer, averages 10.1% active ingredients and 89.9% inactive ingredients; Fizzical, averages 20.1% active ingredients and 79.9% inactive ingredients; Emergen-C Chewable, averages 31% active ingredients and 69% inactive ingredients and sugars; and ODTs (sold over the counter), no more than 10% active ingredients and at least 90% inactive ingredients, sugars and artificial fillers.

Currently over 50% of all U.S. households, and a significantly larger number of households and individuals globally, use daily nutritional supplements in powder-mixes, capsules, tablets and other forms. Given the limitations in convenience, consistency, and effectiveness with respect to the production, use and quantity of active ingredient delivered by currently available systems, new compositions, products, and methods for use in active ingredient delivery are necessary.

### BRIEF DESCRIPTION

In one aspect, this disclosure relates to an intraorally absorbable composition, comprising a) 50-90% of one or more active ingredients; and b) 10-50% of an excipient blend comprising a lubricant; a binder selected from chicory root, a chicory root extract, inulin, or combinations thereof; and devoid or substantially devoid of any other binder, wherein the composition is in a compressed, powdered lozenge form, and wherein the composition fully orally disintegrates within about 1-20 minutes upon oral administration to a subject.

In another aspect, this disclosure relates to a method of making intraorally absorbable compositions, comprising: combining 50-90% of one or more active ingredients with 10-50% of an excipient blend comprising a lubricant; a binder selected from chicory root, a chicory root extract, inulin, or combinations thereof; and devoid or substantially devoid of any other binder; and compressing the combination of the one or more active ingredients and the excipient blend at high pressure and low speed into a compressed, powdered lozenge form that fully orally disintegrates within about 1-20 minutes upon oral administration to a subject.

In another aspect, this disclosure relates to a method of increasing absorption of one or more active ingredients, the method comprising: receiving an intraorally absorbable composition comprising the one or more active ingredients; inserting the composition into the mouth of a subject; and allowing the composition to fully dissolve in the mouth of the subject.

### BRIEF DESCRIPTION OF THE DRAWINGS

The FIGURES described herein depict primarily generalized embodiments or data in support of generalized embodiments, which embodiments and data will be described with additional specificity and detail in connection with the drawings in which:

FIG. 1A is a bar chart comparing absorption and bioavailability of dextrose over baseline after subjects were either dosed intraorally with a solid intraoral formulation (left columns) or dosed orally with a liquid oral formulation (right columns).

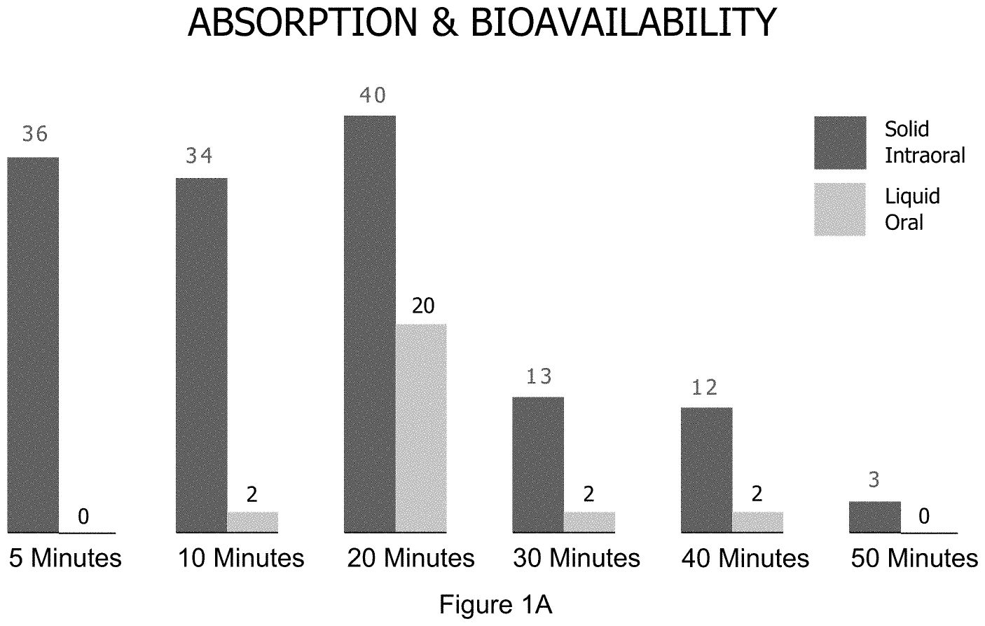

FIG. 1B is a graphical representation of FIG. 1A.

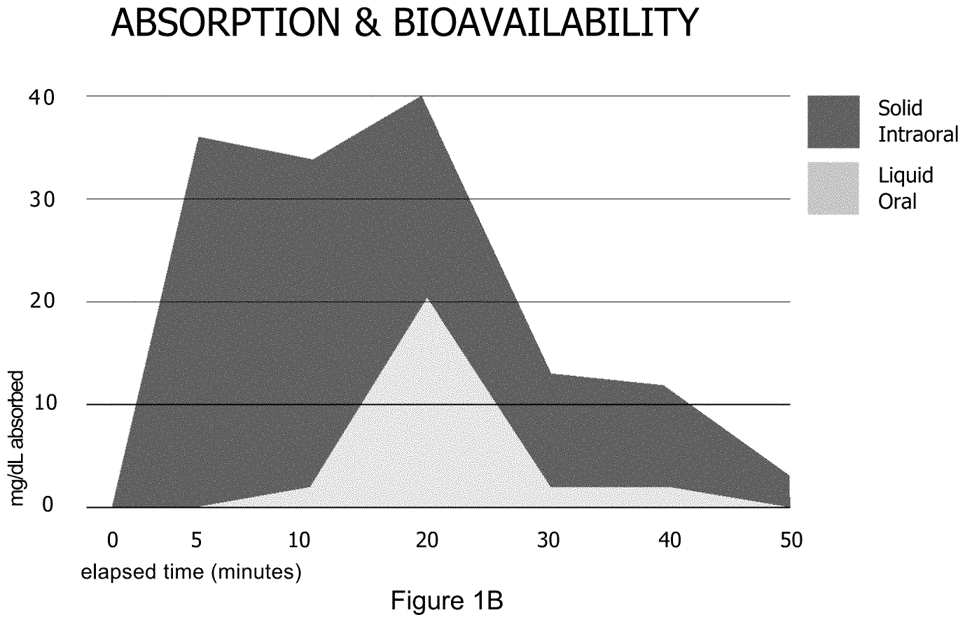

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified unless the context clearly dictates otherwise. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “subject” refers to a warm-blooded animal such as a mammal, which is to benefit from the administration of an active ingredient. It is understood that at least humans, dogs, cats, and horses are within the scope of the meaning of the term. In some embodiments, the subject is human. Generally, as used herein, the term “subject” means a human or an animal for which the compositions of the disclosure may be administered.

As used herein, the term “active ingredient” refers to any pharmaceutical agents, therapeutic substances and/or nutritional substances that may be delivered orally and/or intraorally. Examples of suitable active ingredients include but are not limited to pharmaceutical agents, nutraceutical agents, and nutrients.

As used herein, the term “pharmaceutical agents” refers to any diagnostic or therapeutic drug or combination of drugs that has the property of assisting in the diagnosis, prevention, treatment, cure, or mitigation of abnormal conditions, diseases, or symptoms in a subject. Examples of suitable pharmaceutical agents include but are not limited to drugs classified as either requiring a prescription or available over-the-counter without a prescription; classified as either small-molecule drugs (which may be derived by chemical synthesis) and biopharmaceuticals (including recombinant proteins, vaccines, blood products, gene therapy, monoclonal antibodies and cell therapies); and active substances that are classified by the Anatomical Therapeutic Chemical Classification System (ATC system) (www.who.int/tools/atc-ddd-toolkit/atc-classification).

As used herein, the term “nutraceutical agents” refers to any compounds that act as a pharmaceutical agent alternative that claims at least one physiological benefit when administered to a subject. Examples of suitable nutraceutical agents include but are not limited to prebiotics, probiotics, phytonutrients, hydrolyzed and other proteins and amino acids, polyunsaturated fatty acids, antioxidants, dietary fibers, dietary supplements, vitamins, minerals and electrolytes, stimulants, herbal products, and combinations thereof, whether prepared naturally, artificially, or synthetically.

As used herein, the term “nutrients” refers to any substance that either provide energy, act as building blocks for growth and/or repair, and/or act to regulate chemical processes in a subject. Examples of suitable nutrients include but are not limited to carbohydrates, lipids and fatty acids, proteins and amino acids, vitamins, minerals and electrolytes, stimulants, herbal products, and combinations thereof, whether prepared naturally, artificially, or synthetically.

As used herein, the term “excipient blend” refers to a mixture comprising one or more binders and/or one or more lubricants. Excipient blends may further include additional ingredients including but not limited to flavoring agents, sweeteners, fillers, colorants, stabilizers, preservatives, solvents, glidants, diluents, and disintegrants.

As used herein, the term “excipient profile” refers to both the qualitative and quantitative nature of included binders, lubricants, and other ingredients in an excipient blend. Examples of suitable excipients include but are not limited to the examples of suitable binders, lubricants, flavoring agents, and sweeteners provided herein.

As used herein, the term “binder” refers to substances that are added to powdered particles, generally prior to granulation or direct compression, to achieve the requisite flow property and/or compressibility necessary for effective compression of the powdery particles and/or granules into a tablet or lozenge including a compressed, powdered lozenge form or to improve certain physical properties of the powdered particles including but not limited to increasing the cohesive nature of the powdered particles in forming granules, tablets, lozenges including compressed, powdered lozenges, and other solid dosage forms. In certain embodiments, binders other than chicory root, a chicory root extract, inulin, or combinations thereof or excluded from the compositions. Examples of excluded binders include but are not limited to cellulose and cellulose derivatives (e.g. microcrystalline cellulose, methylcellulose (MC), sodium carboxymethyl cellulose (CMC), hydroxypropyl methylcellulose (HPMC, hypromellose), hydroxyethyl cellulose (HEC), and hydroxypropylcellulose (HPC)); cellulose ethers; polymers (e.g. like polyvinylpyrrolidone (PVP, povidone), polyvinyl alcohol (PVA), polyacrylamides, poly-methyacrylamides, polyoxazolines (POZ), polyphosphates, and polyethylene glycol (PEG)); copolymers (e.g. divinyl ether-maleic anhydride); starches and their derivatives (including pregelatinized and granulated starches, dextrin, and maltodextrin); sugars (e.g. glucose, dextrose, and lactose); sugar alcohols (e.g. mannitol, sorbitol, xylitol, erythritol, maltitol, and isomalt); vegetable waxes (e.g. camauba wax), gelatins and gelatin-like products (e.g. agar); pectins; oligosaccharides (other than inulin), polysaccharides other than inulin (e.g. xanthan gum); and dietary fiber other than chicory root and chicory root extracts (e.g. acacia and guar gum) (www.pharmaexcipients.com/binder/) and (www.naturalproductsinsider.com/contract-manufacturing/binder-selection-tableting).

As used herein, the term “lubricant” refers to ingredients that are generally used to reduce the friction between the tablet and/or lozenge including a compressed, powdered lozenge and the die metal surfaces used in preparing the same, which allows for more efficient tablet and/or lozenge including a compressed, powdered lozenge ejection without cracking or breaking. Examples of suitable lubricants that have been identified include but are not limited to metallic salts of fatty acids (e.g. magnesium stearate, calcium stearate, and zinc stearate), fatty acids (e.g. stearic acid, palmitic acid, and myristic acid), fatty acid esters (e.g. glyceryl monostearate, glyceryl tribehenate, and glyceryl dibehenate), sugar esters (e.g. sorbitan monostearate and sucrose monopalmitate), organic esters (e.g. sodium stearyl fumarate), inorganic materials (e.g. hydrated magnesium silicate, and talc), and polymers (e.g. polyethylene glycol).

As used herein, the term “sweetener” refers to sugars, sugar derivatives, sugar substitutes, and sugar alcohols, including natural and synthetic non-sugar sweeteners. Examples of suitable sweeteners that have been identified include but are not limited to glucose, dextrose, maltose, and lactose, mannitol, sorbitol, xylitol, erythritol, maltitol, isomalt, and sucralose.

As used herein, the term “flavoring agent” refers generally to ingredients that may be comprised of one or more compounds that add an aroma and/or a taste or mask a taste in the composition. Flavoring agents may include natural and/or artificial ingredients. Examples of suitable flavoring agents that have been identified include but are not limited to flavors as categorized by the Code of Federal Regulations (21 CFR § 101.22) into four types including natural flavors, natural with other natural flavors, artificial flavors, and natural and artificial flavors (www.pharmaexcipients.com/what-are-flavors/).

As used herein, the term “intraoral formulation” refers to a prepared composition that is delivered to the mouth and provides the enhanced ability for active ingredients to be absorbed primarily through the mucosa of the mouth upon administration to a subject. Examples of suitable intraoral formulations that have been identified include but are not limited to compositions comprising orally disintegrating tablets, films and lozenges including compressed, powdered lozenges; rapidly disintegrating tablets, films, and lozenges including compressed, powdered lozenges; chewable tablets, films, and lozenges including compressed, powdered lozenges; orally disintegrating powders and granules; liquid solutions, suspensions and sprays; impregnated patches; and other compositions and delivery mechanisms that are designed to produce enhanced delivery of active ingredients through the mucosa of the mouth of a subject.

As used herein, the term “solid intraoral dosage form” refers to intraoral formulation compositions that are in solid form. Examples of suitable solid intraoral dosage forms include but are not limited to compositions comprising orally disintegrating tablets, films and lozenges including compressed, powdered lozenges; rapidly disintegrating tablets, films, and lozenges including compressed, powdered lozenges; chewable tablets, films, and lozenges including compressed, powdered lozenges; orally disintegrating powders and granules; impregnated patches; and other compositions and delivery mechanisms that are in solid form and designed to produce enhanced delivery of active ingredients through the mucosa of the mouth of a subject. The present disclosure is directed to novel solid nutrient delivery products and compositions and methods for use in producing the same that reduce and/or eliminate inconveniences, inconsistency, and ineffectiveness regarding both the use and quantity of active ingredient delivery compared to currently available delivery methods. Subjects that administer active ingredients, including nutritional supplements and other health products, for reasons including enhancing health, improving athletic performance, and supplementing their diet can benefit from the convenience, efficiency, higher nutrient density and enhanced bioavailability of active ingredients in products and compositions of the present disclosure.

The present disclosure allows for a higher density of active ingredients in powdered and solid intraoral dosage forms that allows for administration of a smaller unit of nutrient delivery products and compositions, or provides a larger dose of active ingredients in the same size unit compared to currently available delivery methods. The present disclosure also provides for delivery forms that can provide enhanced digestive absorption as well as the benefits of intraoral administration that protects active ingredients from being degraded in the harsh gut environment and exposed to hepatic first-pass degradation and gastric emptying processes, each of which can limit absorption and systemic circulation.

Solid nutrient delivery, administered intraorally, provides a higher rate and overall amount of nutrient absorption by leveraging increased intraoral absorption and increased digestive absorption through the natural chewing, sucking, salivary scavenging and stimulation of digestive enzyme production attendant to the consumption of the products and compositions of the present disclosure.

It is believed that intraoral delivery of active ingredients is a superior method of administration because absorption through the mucosal linings of the oral cavity pass the active ingredients directly into the systemic circulation through the jugular vein, thus avoiding passage through the liver where they may undergo undesirable first-pass metabolism. Intraoral delivery also benefits from the avoidance of presystemic elimination and degradation in the hostile GI tract. Additional tangible benefits of intraoral delivery include increased levels of absorption, faster onset of action and greater bioavailability. Furthermore, intraoral delivery does not require swallowing and does not produce gastrointestinal irritation. (life-enhancement.com/pages/intraoral-delivery-is-a-better-route).

Bioavailability is a key determination in considering deliver forms for administration of active ingredients. A common misconception is that active ingredients are best absorbed when they are delivered in solution. (academic.oup.com/jn/article/131/4/1349S/4686865). Rather, ingested fluids are initially stored in the stomach, where there is generally little net absorption of water or solute across the gastric mucosa.” (academic.oup.com/nutritionreviews/article/73/suppl_2/57/1930269). Also, active ingredients administered perorally in tablet or capsule form are first digested in the gut, which often results in unnecessary degradation. (www.psychologytoday.com/us/blog/food-junkie/201810/what-you-need-know-about-sublingual-vitamins).

Active ingredients that are swallowed must also survive exposure to low pH acids in the stomach and numerous GI secretions, including potentially degrading enzymes. The remaining active ingredients must then be absorbed by transport across membranes of the epithelial cells in the GI tract, which transport can be affected by the presence of a thick mucus layer in the stomach, differences in luminal pH along the GI tract, the limited surface area of epithelium per luminal volume, blood perfusion, the presence of bile, the nature of epithelial membranes, and first-pass metabolism. (www.merckmanuals.com/professional/clinical-pharmacology/pharmacokinetics/drug-absorption).

First-pass metabolism is a phenomenon that occurs because ingredients absorbed through the vasculature of the intestinal lining must first pass via the portal vein through the liver, which is a major site of drug metabolism, before they are released to the systemic circulation. (www.ncbi.nlm.nih.gov/books/NBK551679/). As a result, in some cases only a small proportion of the active drug reaches the systemic circulation and its intended target tissue. (www.slideshare.net/BUDDHABHUSHANO/first-pass-metabolism-buddhabhushan-dongre).

The oral mucosa has a thin epithelium and rich vascularity, favoring absorption; however, active ingredients generally must be in contact with the oral mucosa longer than normal chewing and swallowing allows for substantial absorption to occur. Some methods that enhance intraoral delivery and absorption of active ingredients include placing products between the gums and cheek (buccal administration), under the tongue (sublingual administration), allowing dissolution in the oral cavity, and/or involve prolonged chewing. (www.merckmanuals.com/professional/clinical-pharmacology/pharmacokinetics/drug-absorption), (www.ncbi.nlm.nih.gov/pubmed/12126458), (www.nottingham.ac.uk/nmp/sonet/rlos/bioproc/metabolism/02.html). Studies have demonstrated that the amount of intraoral absorption of some drugs through the sublingual route is 3×-10× greater than traditional peroral administration, results that were only surpassed by hypodermic injection. (innovareacademics.in/joumal/ijpps/Vol3Suppl2/1092.pdf). As a result, intraoral administration allows for administration of lower doses of active ingredients to achieve the same levels in the systemic circulation. (www.healthline.com/health/sublingual-and-buccal-medication-administration).

Unlike other single and multi-ingredient solid nutrient delivery products existing in the market, certain embodiments disclosed herein allow for dramatically enhanced flavor profiles because of a dramatically reduced excipient profile requirement. Certain embodiments also allow for the elimination of sugars, sugar derivatives, sugar substitutes, and sugar alcohols as part of the production and manufacturing process by including soluble fibers.

The solid intraoral delivery products and compositions and methods for use in producing the same, which are disclosed for the present embodiment, are notable for providing higher nutrient density and bioavailability, thus allowing smaller unit size to reach the same active ingredient levels in the systemic circulation, or similar unit size to reach improved active ingredient levels, over existing delivery methods.

It is believed that manufacturing a solid intraoral dosage form product originating from a powdered form has not been demonstrated and is not possible without the use of a binding agent. For example, tablet binders, sometimes referred to as adhesives, are one of the most essential elements in the formulation of a tablet. Tablet and lozenge binders, including compressed, powdered lozenge binders, function by promoting cohesiveness and aiding the mixing of other ingredients in a tablet. Tablet binders are sometimes used to turn powder into granules, which is achieved through the process of granulation, or to prepare powders for direct compression. During granulation, powder substances are accumulated with the aid of a binder to form larger particles called granules (www.lfatabletpresses.com/articles/tablet-binders). Binders also are believed to play a vital role in making sure pellets or granules, tablets, and lozenges including compressed, powdered lozenges remain in shape until they reach their target by holding all ingredients (active ingredients and an excipient blend) together in a solid dosage form (pharmaeducation.net/tablet-binder/). In this regard, binders can be used to overcome many of the challenges of direct compression performed in the absence of an effective binder for achieving desirable tablet or lozenge, including a compressed, powdered lozenge, mechanical properties, such as strength and friability from poorly compressible high-dose active ingredients, including pharmaceutical, nutraceutical, and nutrient active ingredients (www.naturalproductsinsider.com/contract-manufacturing/binder-selection-tableting).

The embodiments herein take advantage of the properties of dietary fibers as binders in the preparation of solid active ingredient delivery products and compositions. Examples of dietary fibers include inulin and chicory root and chicory root extracts. Inulin is a water-soluble storage oligosaccharide/polysaccharide and belongs to a group of non-digestible carbohydrates called fructans (pubmed.ncbi.nlm.nih.gov/27178951/). Inulin is a type of fiber, meaning it's a plant-based carbohydrate whose bonds cannot be broken by human digestive enzymes. Like all fibers, dietary inulin is not digested in the small intestine but rather travels intact to the colon where it is generally digested by resident bacteria before being passed in stools (health.usnews.com/health-news/blogs/eat-run/2015/05/05/what-is-inulin-chicory-root-fiberz). Inulin can be used to replace dietary fats and/or sugars in some processed and functional foods (www.verywellfit.com/the-health-benefits-of-chicory-root-4178997). Inulin has been consumed by humans since antiquity. People on all five inhabited continents have eaten various roots and tubers, some containing starch and some containing inulin or a similar glucofructan. Inulin has been reported to have a sweetening power of 30-65% that of sucrose and a high degree of polymerization (DP) of 2-60. A major source of inulin is chicory root (www.sciencedirect.com/topics/food-science/chicory-roots). Due to the high content of inulin, chicory root is generally classified as a fiber rather than a starch (www.eatthis.com/chicory-root/). In fact, the FDA has determined that inulin-type fructans derived from chicory root are dietary fiber for the new nutrition facts label (www.bakemag.com/articles/8717-fda-confirms-dietary-fiber-status-for-chicory-root-fiber).

In the embodiments disclosed herein, it was an unexpected discovery that active ingredients combined using the disclosed methods with an excipient blend comprising inulin, chicory root, chicory root extracts, or combinations thereof with lubricants allows for the manufacture of intraoral formulation products and compositions with beneficial improved properties to those currently available. For example, the embodiments disclosed herein can include active ingredients at significantly higher amounts and density, the excipient content can be substantially reduced, the need for sugars, sugar derivatives, sugar substitutes, and sugar alcohols can be eliminated, manufacture and administration can be completed more conveniently and efficiently, and enhanced bioavailability can be achieved.

In various embodiments, the compositions of the disclosure include solid nutrient delivery products and compositions and methods for use in producing the same. In various embodiments, the compositions of the disclosure include powdered forms comprising one or more active ingredients combined with an excipient blend comprising one or more binders and/or one or more lubricants. In various embodiments, the compositions of the disclosure include a solid oral dosage forms comprising one or more active ingredients combined with an excipient blend comprising one or more binders and/or one or more lubricants. In various embodiments, suitable binders may include dietary fibers. In various embodiments, the dietary fibers may include inulin, chicory root, and/or chicory root extracts. In various embodiments, suitable lubricants may include magnesium stearate, stearic acid or combinations thereof.

In certain embodiments, the compositions will be manufactured in powdered form. Preferentially, the powdered form of the compositions will be comprised of mixtures of active ingredients and an excipient blend that can be effectively compressed into solid oral dosage forms. Preferentially, the powdered form of the compositions will be comprised of mixtures of active ingredients and an excipient blend that can be effectively compressed into solid oral dosage forms that are lozenges including compressed, powdered lozenges. Preferentially, the powdered form of the compositions will be comprised of mixtures of active ingredients and an excipient blend that can be effectively compressed into solid oral dosage forms that are lozenges including compressed, powdered lozenges that provide for efficient bioavailability of active ingredients by intraoral absorption.

In various embodiments, the compositions will be manufactured in solid oral dosage form. Preferentially, the solid oral dosage form of the compositions will be comprised of mixtures of active ingredients and an excipient blend. Preferentially, the solid oral dosage form of the compositions will be comprised of mixtures of active ingredients and an excipient blend that are lozenges including compressed, powdered lozenges. Preferentially, the solid oral dosage form of the compositions will be comprised of mixtures of active ingredients and an excipient blend that are lozenges including compressed, powdered lozenges that provide for efficient bioavailability of active ingredients by intraoral absorption.

Preferentially, the compositions of the disclosure will contain high amounts and high density of active ingredients.

Preferentially, the compositions of the disclosure will contain low amounts of excipient ingredients.

Preferentially, the compositions of the disclosure can be produced without the need for sugars, sugar derivatives, sugar substitutes, and sugar alcohols.

Preferentially, the compositions of the disclosure will allow for high bioavailability after administration to a subject.

In various embodiments, the compositions may include an effective amount of active ingredients. Suitable active ingredients may include pharmaceutical agents, nutraceutical agents, and/or nutrients. In various embodiments, the compositions of the disclosure may include an effective amount of at least about 30 wt % of one or more active ingredients, or between about 30 wt % to about 95 wt %, or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 wt %, or any range between any two of these amounts including about 30 wt % to about 40 wt %, or about 30 wt % to about 50 wt %, or about 30 wt % to about 60 wt %, or about 30 wt % to about 70 wt %, or about 30 wt % to about 80 wt %, or about 30 wt % to about 90 wt %, or about 40 wt % to about 50 wt %, or about 40 wt % to about 60 wt %, or about 40 wt % to about 70 wt %, or about 40 wt % to about 80 wt %, or about 40 wt % to about 90 wt %, or about 50 wt % to about 60 wt %, or about 50 wt % to about 70 wt %, or about 50 wt % to about 80 wt %, or about 50 wt % to about 90 wt %, or about 60 wt % to about 70 wt %, or about 60 wt % to about 80 wt %, or about 60 wt % to about 90 wt %, or about 70 wt % to about 80 wt %, or about 70 wt % to about 90 wt %, or about 80 wt % to about 90 wt % of one or more active ingredients. In some preferred forms, the amount of one or more active ingredients is sufficient to maintain an optimum level of one or more active ingredients in the systemic circulation of a subject receiving an administration of the composition on a daily basis.

In various embodiments, the compositions may include an amount of an excipient blend. Suitable excipient blends are comprised of one or more suitable binders and/or one or more suitable lubricants. In various embodiments, the compositions of the disclosure may include an amount of at least about 5 wt % of an excipient blend, or between about 5 wt % to about 70 wt %, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 5 wt % to about 10 wt %, or about 5 wt % to about 20 wt %, or about 5 wt % to about 30 wt %, or about 5 wt % to about 40 wt %, or about 5 wt % to about 50 wt %, or about 5 wt % to about 60 wt %, or about 5 wt % to about 70 wt %, or about 10 wt % to about 20 wt %, or about 10 wt % to about 30 wt %, or about 10 wt % to about 40 wt %, or about 10 wt % to about 50 wt %, or about 10 wt % to about 60 wt %, or about 10 wt % to about 70 wt %, or about 15 wt % to about 20 wt %, or about 15 wt % to about 30 wt %, or about 15 wt % to about 40 wt %, or about 15 wt % to about 50 wt %, or about 15 wt % to about 60 wt %, or about 15 wt % to about 70 wt %, or about 20 wt % to about 30 wt %, or about 20 wt % to about 40 wt %, or about 20 wt % to about 50 wt %, or about 20 wt % to about 60 wt %, or about 20 wt % to about 70 wt %, or about 30 wt % to about 40 wt %, or about 30 wt % to about 50 wt %, or about 30 wt % to about 60 wt %, or about 30 wt % to about 70 wt %, or about 40 wt % to about 50 wt %, or about 40 wt % to about 60 wt %, or about 40 wt % to about 70 wt %, or about 50 wt % to about 60 wt %, or about 50 wt % to about 70 wt %, or about 60 wt % to about 70 wt % of an excipient blend.

In various embodiments, the compositions may include an amount one or more binders. Suitable binders may include chicory root, a chicory root extract, inulin, or combinations thereof. In various embodiments, the compositions of the disclosure may include an amount of at least about 1 wt % of one or more binders, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more binders. Preferably, the composition includes about 1 wt % to about 10 wt % of a binder selected from chicory root, a chicory root extract, inulin, or combinations thereof, and devoid or substantially devoid of any other binder.

In various embodiments, the compositions may be devoid of or substantially devoid of additional binders other than chicory root, a chicory root extract, inulin, or combinations thereof. The term “substantially devoid” refers to amounts that are less than 5 wt % of the composition, or between about 0 wt % to about 5 wt %, or about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt %, or any range between any two of these amounts including about 0 wt % to about 0.01 wt %, or about 0 wt % to about 0.05 wt %, or about 0 wt % to about 0.01 wt %, or about 0 wt % to about 1 wt %, or about 0 wt % to about 2.5 wt %, or about 0 wt % to about 5 wt %, or about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.1 wt % to about 1 wt %, or about 0.1 wt % to about 2.5 wt %, or about 0.1 wt % to about 5 wt %, or about 0.5 wt % to about 1 wt %, or about 0.5 wt % to about 2.5 wt %, or about 0.5 wt % to about 5 wt %, or about 1 wt % to about 2.5 wt %, or about 1 wt % to about 5 wt %, or about 2 wt % to about 5 wt %, or about 3 wt % to about 5 wt %, or about 4 wt % to about 5 wt % of the composition.

In various embodiments, the compositions may include an amount one or more lubricants. Suitable lubricants may include magnesium stearate, stearic acid or combinations thereof. In various embodiments, the compositions of the disclosure may include no lubricants. In various embodiments, the compositions of the disclosure may include an amount of at least about 0.5 wt % of one or more lubricants, or between about 0.5 wt % to about 30 wt %, or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 wt %, or any range between any two of these amounts including about 0.5 wt % to about 1 wt %, or about 0.5 wt % to about 2 wt %, or about 0.5 wt % to about 3 wt %, or about 0.5 wt % to about 4 wt %, or about 0.5 wt % to about 5 wt %, or about 0.5 wt % to about 10 wt %, or about 1 wt % to about 2 wt %, or about 1 wt % to about 3 wt %, or about 1 wt % to about 4 wt %, or about 1 wt % to about 5 wt %, or about 1 wt % to about 10 wt %, or about 1 wt % to about 15 wt %, or about 1 wt % to about 20 wt %, or about 1 wt % to about 25 wt %, or about 1 wt % to about 30 wt %, or about 2 wt % to about 5 wt %, or about 2 wt % to about 10 wt %, or about 2 wt % to about 15 wt %, or about 2 wt % to about 20 wt %, or about 2 wt % to about 25 wt %, or about 2 wt % to about 30 wt %, or about 3 wt % to about 5 wt %, or about 3 wt % to about 10 wt %, or about 3 wt % to about 15 wt %, or about 3 wt % to about 20 wt %, or about 3 wt % to about 25 wt %, or about 3 wt % to about 30 wt %, or about 4 wt % to about 5 wt %, or about 4 wt % to about 10 wt %, or about 4 wt % to about 15 wt %, or about 4 wt % to about 20 wt %, or about 4 wt % to about 25 wt %, or about 4 wt % to about 30 wt %, or about 5 wt % to about 10 wt %, or about 5 wt % to about 15 wt %, or about 5 wt % to about 20 wt %, or about 5 wt % to about 25 wt %, or about 5 wt % to about 30 wt %, or about 10 wt % to about 15 wt %, or about 10 wt % to about 20 wt %, or about 10 wt % to about 25 wt %, or about 10 wt % to about 30 wt %, or about 15 wt % to about 20 wt %, or about 15 wt % to about 25 wt %, or about 15 wt % to about 30 wt %, or about 20 wt % to about 25 wt %, or about 20 wt % to about 30 wt %, or about 25 wt % to about 30 wt % of one or more lubricants.

In various embodiments, the compositions may further include one or more flavoring agents. Suitable flavoring agents may include natural flavors, natural with other natural flavors, artificial flavors, and natural and artificial flavors. In various embodiments, the compositions of the disclosure may include no flavoring agents. In various embodiments, the compositions of the disclosure may include an amount of at least about 1 wt % of one or more flavoring agents, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more flavoring agents.

In various embodiments, the compositions may further include one or more sweeteners. Suitable sweeteners may include sugars, sugar derivatives, sugar substitutes, and sugar alcohols. In various embodiments, the compositions of the disclosure may include no sweeteners or substantially no sweeteners. In various embodiments, the compositions of the disclosure may include an amount of at least about 0.01 wt % of one or more sweeteners, or between about 0.01 wt % to about 10 wt %, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt %, or any range between any two of these amounts including about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.01 wt % to about 10 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.03 wt % to about 10 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.05 wt % to about 10 wt %, or about 0.1 wt % to about 1 wt %, or about 0.1 wt % to about 2.5 wt %, or about 0.1 wt % to about 5 wt %, or about 0.1 wt % to about 10 wt %, or about 0.5 wt % to about 1 wt %, or about 0.5 wt % to about 2.5 wt %, or about 0.5 wt % to about 5 wt %, or about 0.5 wt % to about 10 wt %, or about 1 wt % to about 2.5 wt %, or about 1 wt % to about 5 wt %, or about 1 wt % to about 10 wt %, or about 2 wt % to about 5 wt %, or about 2 wt % to about 10 wt %, or about 3 wt % to about 5 wt %, or about 3 wt % to about 10 wt %, or about 4 wt % to about 10 wt %, or about 5 wt % to about 10 wt % of one or more sweeteners.

In various embodiments, the compositions orally disintegrate upon oral administration to a subject. In various embodiments, the compositions of the disclosure orally disintegrate in less than 30 minutes upon oral administration to a subject, or between about 1 min to about 30 min, or about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or 30 min, or any range between any two of these amounts including about 1 min to about 2 min, or about 1 min to about 3 min, or about 1 min to about 4 min, or about 1 min to about 5 min, or about 1 min to about 7.5 min, or about 1 min to about 10 min, or about 1 min to about 15 min, or about 1 min to about 20 min, or about 1 min to about 25 min, or about 1 min to about 30 min, or about 2 min to about 3 min, or about 2 min to about 4 min, or about 2 min to about 5 min, or about 2 min to about 7.5 min, or about 2 min to about 10 min, or about 2 min to about 15 min, or about 2 min to about 20 min, or about 2 min to about 25 min, or about 2 min to about 30 min, or about 3 min to about 4 min, or about 3 min to about 5 min, or about 3 min to about 7.5 min, or about 3 min to about 10 min, or about 3 min to about 15 min, or about 3 min to about 20 min, or about 3 min to about 25 min, or about 3 min to about 30 min, or about 4 min to about 5 min, or about 4 min to about 7.5 min, or about 4 min to about 10 min, or about 4 min to about 15 min, or about 4 min to about 20 min, or about 4 min to about 25 min, or about 4 min to about 30 min, or about 5 min to about 7.5 min, or about 5 min to about 10 min, or about 5 min to about 15 min, or about 5 min to about 20 min, or about 5 min to about 25 min, or about 5 min to about 30 min, or about 7.5 min to about 10 min, or about 7.5 min to about 15 min, or about 7.5 min to about 20 min, or about 7.5 min to about 25 min, or about 7.5 min to about 30 min, or about 10 min to about 15 min, or about 10 min to about 20 min, or about 10 min to about 25 min, or about 10 min to about 30 min, or about 15 min to about 20 min, or about 15 min to about 25 min, or about 15 min to about 30 min, or about 20 min to about 25 min, or about 20 min to about 30 min, or about 25 min to about 30 min upon oral administration to a subject.

Various embodiments of the disclosure further relate to methods of making oral formulations with a decreased excipient profile, comprising adding an amount of one or more active ingredients to an amount of an excipient blend comprising one or more binders and/or one or more lubricants to produce a powdered composition. Various embodiments of the disclosure further relate to methods of making oral formulations with a decreased excipient profile, comprising adding an amount of one or more active ingredients to an amount of an excipient blend comprising one or more binders and/or one or more lubricants to produce a solid oral dosage form. In various embodiments, the dietary fibers may include inulin, chicory root, and/or chicory root extracts. In various embodiments, suitable lubricants may include magnesium stearate, stearic acid or combinations thereof.

In various embodiments, the methods include making oral formulations of the compositions in powdered form. Preferentially, the methods include making oral formulations of powdered forms of the composition comprised of mixtures of active ingredients and an excipient blend that can be effectively compressed into solid oral dosage forms. Preferentially, the methods include making oral formulations of powdered forms of the composition comprised of mixtures of active ingredients and an excipient blend that can be effectively compressed into solid oral dosage forms that are lozenges including compressed, powdered lozenges. Preferentially, the methods include making oral formulations of powdered forms of the compositions comprised of mixtures of active ingredients and an excipient blend that can be effectively compressed into solid oral dosage forms that are lozenges including compressed, powdered lozenges that provide for efficient bioavailability of active ingredients by intraoral absorption.

In various embodiments, the methods include making oral formulations of compositions manufactured in solid oral dosage form. Preferentially, the methods include making oral formulations of solid oral dosage forms of the compositions comprised of mixtures of active ingredients and an excipient blend. Preferentially, the methods include making oral formulations of solid oral dosage forms of the compositions comprised of mixtures of active ingredients and an excipient blend that are lozenges including compressed, powdered lozenges. Preferentially, the methods include making oral formulations of solid oral dosage forms of the compositions comprised of mixtures of active ingredients and an excipient blend that are lozenges including compressed, powdered lozenges that provide for efficient bioavailability of active ingredients by intraoral absorption.

Preferentially, the methods include making oral formulations of the compositions that contain high amounts and high density of active ingredients.

Preferentially, the methods include making oral formulations of the compositions of that contain low amounts of excipient ingredients.

Preferentially, the methods include making oral formulations of the compositions that can be produced without the need for sugars, sugar derivatives, sugar substitutes, and sugar alcohols.

Preferentially, the methods include making oral formulations of the compositions that allow for high bioavailability after administration to a subject.

In various embodiments, the methods include making oral formulations of the compositions that may include an effective amount of active ingredients. Suitable active ingredients may include pharmaceutical agents, nutraceutical agents, and/or nutrients. In various embodiments, the methods include making oral formulations of the compositions that may include an effective amount of at least about 30 wt % of one or more active ingredients, or between about 30 wt % to about 95 wt %, or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 wt %, or any range between any two of these amounts including about 30 wt % to about 40 wt %, or about 30 wt % to about 50 wt %, or about 30 wt % to about 60 wt %, or about 30 wt % to about 70 wt %, or about 30 wt % to about 80 wt %, or about 30 wt % to about 90 wt %, or about 40 wt % to about 50 wt %, or about 40 wt % to about 60 wt %, or about 40 wt % to about 70 wt %, or about 40 wt % to about 80 wt %, or about 40 wt % to about 90 wt %, or about 50 wt % to about 60 wt %, or about 50 wt % to about 70 wt %, or about 50 wt % to about 80 wt %, or about 50 wt % to about 90 wt %, or about 60 wt % to about 70 wt %, or about 60 wt % to about 80 wt %, or about 60 wt % to about 90 wt %, or about 70 wt % to about 80 wt %, or about 70 wt % to about 90 wt %, or about 80 wt % to about 90 wt % of one or more active ingredients. In some preferred forms, the methods include making oral formulations wherein the amount of one or more active ingredients is sufficient to maintain an optimum level of one or more active ingredients in the systemic circulation of a subject receiving an administration of the composition on a daily basis.

In various embodiments, the methods include making oral formulations of the compositions that may include an amount of an excipient blend. Suitable excipient blends are comprised of one or more suitable binders and/or one or more suitable lubricants. In various embodiments, the methods include making oral formulations of the compositions that may include an amount of at least about 5 wt % of an excipient blend, or between about 5 wt % to about 70 wt %, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 5 wt % to about 10 wt %, or about 5 wt % to about 20 wt %, or about 5 wt % to about 30 wt %, or about 5 wt % to about 40 wt %, or about 5 wt % to about 50 wt %, or about 5 wt % to about 60 wt %, or about 5 wt % to about 70 wt %, or about 10 wt % to about 20 wt %, or about 10 wt % to about 30 wt %, or about 10 wt % to about 40 wt %, or about 10 wt % to about 50 wt %, or about 10 wt % to about 60 wt %, or about 10 wt % to about 70 wt %, or about 15 wt % to about 20 wt %, or about 15 wt % to about 30 wt %, or about 15 wt % to about 40 wt %, or about 15 wt % to about 50 wt %, or about 15 wt % to about 60 wt %, or about 15 wt % to about 70 wt %, or about 20 wt % to about 30 wt %, or about 20 wt % to about 40 wt %, or about 20 wt % to about 50 wt %, or about 20 wt % to about 60 wt %, or about 20 wt % to about 70 wt %, or about 30 wt % to about 40 wt %, or about 30 wt % to about 50 wt %, or about 30 wt % to about 60 wt %, or about 30 wt % to about 70 wt %, or about 40 wt % to about 50 wt %, or about 40 wt % to about 60 wt %, or about 40 wt % to about 70 wt %, or about 50 wt % to about 60 wt %, or about 50 wt % to about 70 wt %, or about 60 wt % to about 70 wt % of an excipient blend.

In various embodiments, the methods include making oral formulations of the compositions that may include an amount one or more binders. Suitable binders may include chicory root, a chicory root extract, inulin, or combinations thereof. In various embodiments, the methods include making oral formulations of the compositions that may include an amount of at least about 1 wt % of one or more binders, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more binders. Preferably, the methods include making oral formulations of the composition that include about 1 wt % to about 10 wt % of a binder selected from chicory root, a chicory root extract, inulin, or combinations thereof, and devoid or substantially devoid of any other binder.

In various embodiments, the methods include making oral formulations of the compositions that may be devoid of or substantially devoid of additional binders other than chicory root, a chicory root extract, inulin, or combinations thereof. The term “substantially devoid” refers to amounts that are less than 5 wt % of the composition, or between about 0 wt % to about 5 wt %, or about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt %, or any range between any two of these amounts including about 0 wt % to about 0.01 wt %, or about 0 wt % to about 0.05 wt %, or about 0 wt % to about 0.01 wt %, or about 0 wt % to about 1 wt %, or about 0 wt % to about 2.5 wt %, or about 0 wt % to about 5 wt %, or about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.1 wt % to about 1 wt %, or about 0.1 wt % to about 2.5 wt %, or about 0.1 wt % to about 5 wt %, or about 0.5 wt % to about 1 wt %, or about 0.5 wt % to about 2.5 wt %, or about 0.5 wt % to about 5 wt %, or about 1 wt % to about 2.5 wt %, or about 1 wt % to about 5 wt %, or about 2 wt % to about 5 wt %, or about 3 wt % to about 5 wt %, or about 4 wt % to about 5 wt % of the composition.

In various embodiments, the methods include making oral formulations of the compositions that may include an amount one or more lubricants. Suitable lubricants may include magnesium stearate, stearic acid or combinations thereof. In various embodiments, the methods include making oral formulations of the compositions that may include no lubricants. In various embodiments, the methods include making oral formulations of the compositions that may include an amount of at least about 0.5 wt % of one or more lubricants, or between about 0.5 wt % to about 30 wt %, or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 wt %, or any range between any two of these amounts including about 0.5 wt % to about 1 wt %, or about 0.5 wt % to about 2 wt %, or about 0.5 wt % to about 3 wt %, or about 0.5 wt % to about 4 wt %, or about 0.5 wt % to about 5 wt %, or about 0.5 wt % to about 10 wt %, or about 1 wt % to about 2 wt %, or about 1 wt % to about 3 wt %, or about 1 wt % to about 4 wt %, or about 1 wt % to about 5 wt %, or about 1 wt % to about 10 wt %, or about 1 wt % to about 15 wt %, or about 1 wt % to about 20 wt %, or about 1 wt % to about 25 wt %, or about 1 wt % to about 30 wt %, or about 2 wt % to about 5 wt %, or about 2 wt % to about 10 wt %, or about 2 wt % to about 15 wt %, or about 2 wt % to about 20 wt %, or about 2 wt % to about 25 wt %, or about 2 wt % to about 30 wt %, or about 3 wt % to about 5 wt %, or about 3 wt % to about 10 wt %, or about 3 wt % to about 15 wt %, or about 3 wt % to about 20 wt %, or about 3 wt % to about 25 wt %, or about 3 wt % to about 30 wt %, or about 4 wt % to about 5 wt %, or about 4 wt % to about 10 wt %, or about 4 wt % to about 15 wt %, or about 4 wt % to about 20 wt %, or about 4 wt % to about 25 wt %, or about 4 wt % to about 30 wt %, or about 5 wt % to about 10 wt %, or about 5 wt % to about 15 wt %, or about 5 wt % to about 20 wt %, or about 5 wt % to about 25 wt %, or about 5 wt % to about 30 wt %, or about 10 wt % to about 15 wt %, or about 10 wt % to about 20 wt %, or about 10 wt % to about 25 wt %, or about 10 wt % to about 30 wt %, or about 15 wt % to about 20 wt %, or about 15 wt % to about 25 wt %, or about 15 wt % to about 30 wt %, or about 20 wt % to about 25 wt %, or about 20 wt % to about 30 wt %, or about 25 wt % to about 30 wt % of one or more lubricants.

In various embodiments, the methods include making oral formulations of the compositions that may further include one or more flavoring agents. Suitable flavoring agents may include natural flavors, natural with other natural flavors, artificial flavors, and natural and artificial flavors. In various embodiments, the methods include making oral formulations of the compositions that may include no flavoring agents. In various embodiments, the methods include making oral formulations of the compositions that may include an amount of at least about 1 wt % of one or more flavoring agents, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more flavoring agents.

In various embodiments, the methods include making oral formulations of the compositions that may further include one or more sweeteners. Suitable sweeteners may include sugars, sugar derivatives, sugar substitutes, and sugar alcohols. In various embodiments, the methods include making oral formulations of the compositions that may include no sweeteners or substantially no sweeteners. In various embodiments, the methods include making oral formulations of the compositions that may include an amount of at least about 0.01 wt % of one or more sweeteners, or between about 0.01 wt % to about 10 wt %, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt %, or any range between any two of these amounts including about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.01 wt % to about 10 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.03 wt % to about 10 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.05 wt % to about 10 wt %, or about 0.1 wt % to about 1 wt %, or about 0.1 wt % to about 2.5 wt %, or about 0.1 wt % to about 5 wt %, or about 0.1 wt % to about 10 wt %, or about 0.5 wt % to about 1 wt %, or about 0.5 wt % to about 2.5 wt %, or about 0.5 wt % to about 5 wt %, or about 0.5 wt % to about 10 wt %, or about 1 wt % to about 2.5 wt %, or about 1 wt % to about 5 wt %, or about 1 wt % to about 10 wt %, or about 2 wt % to about 5 wt %, or about 2 wt % to about 10 wt %, or about 3 wt % to about 5 wt %, or about 3 wt % to about 10 wt %, or about 4 wt % to about 10 wt %, or about 5 wt % to about 10 wt % of one or more sweeteners.

In various embodiments, the methods include making oral formulations of the compositions that orally disintegrate upon oral administration to a subject. In various embodiments, the methods include making oral formulations of the compositions of the disclosure that orally disintegrate in less than 30 minutes upon oral administration to a subject, or between about 1 min to about 30 min, or about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or 30 min, or any range between any two of these amounts including about 1 min to about 2 min, or about 1 min to about 3 min, or about 1 min to about 4 min, or about 1 min to about 5 min, or about 1 min to about 7.5 min, or about 1 min to about 10 min, or about 1 min to about 15 min, or about 1 min to about 20 min, or about 1 min to about 25 min, or about 1 min to about 30 min, or about 2 min to about 3 min, or about 2 min to about 4 min, or about 2 min to about 5 min, or about 2 min to about 7.5 min, or about 2 min to about 10 min, or about 2 min to about 15 min, or about 2 min to about 20 min, or about 2 min to about 25 min, or about 2 min to about 30 min, or about 3 min to about 4 min, or about 3 min to about 5 min, or about 3 min to about 7.5 min, or about 3 min to about 10 min, or about 3 min to about 15 min, or about 3 min to about 20 min, or about 3 min to about 25 min, or about 3 min to about 30 min, or about 4 min to about 5 min, or about 4 min to about 7.5 min, or about 4 min to about 10 min, or about 4 min to about 15 min, or about 4 min to about 20 min, or about 4 min to about 25 min, or about 4 min to about 30 min, or about 5 min to about 7.5 min, or about 5 min to about 10 min, or about 5 min to about 15 min, or about 5 min to about 20 min, or about 5 min to about 25 min, or about 5 min to about 30 min, or about 7.5 min to about 10 min, or about 7.5 min to about 15 min, or about 7.5 min to about 20 min, or about 7.5 min to about 25 min, or about 7.5 min to about 30 min, or about 10 min to about 15 min, or about 10 min to about 20 min, or about 10 min to about 25 min, or about 10 min to about 30 min, or about 15 min to about 20 min, or about 15 min to about 25 min, or about 15 min to about 30 min, or about 20 min to about 25 min, or about 20 min to about 30 min, or about 25 min to about 30 min upon oral administration to a subject.

Various embodiments of the disclosure further relate to methods of increasing absorption of one or more active ingredients of an oral formulation with a decreased excipient profile. Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject. Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject; wherein the composition comprises a) 50-90% of one or more active ingredients; and b) 10-50% of an excipient blend comprising a lubricant; a binder selected from chicory root, a chicory root extract, inulin, or combinations thereof Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject; wherein the composition comprises a) 50-90% of one or more active ingredients; and b) 10-50% of an excipient blend comprising a lubricant; a binder selected from chicory root, a chicory root extract, inulin, or combinations thereof, and devoid or substantially devoid of any other binder.

Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject, wherein the composition comprises a) 50-90% of one or more active ingredients; and b) 10-50% of an excipient blend comprising a lubricant; a binder selected from chicory root, a chicory root extract, inulin, or combinations thereof; and devoid or substantially devoid of any other binder, and wherein the composition is in a powdered form. Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject, wherein the composition comprises a) 50-90% of one or more active ingredients; and b) 10-50% of an excipient blend comprising a lubricant; a binder selected from chicory root, a chicory root extract, inulin, or combinations thereof; and devoid or substantially devoid of any other binder, and wherein the composition is in a powdered form, and wherein the composition fully orally disintegrates within about 1-20 minutes upon oral administration to a subject. In various embodiments, the dietary fibers may include inulin, chicory root, and/or chicory root extracts. In various embodiments, suitable lubricants may include magnesium stearate, stearic acid or combinations thereof.

Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject, wherein the composition comprises a) 50-90% of one or more active ingredients; and b) 10-50% of an excipient blend comprising a lubricant; a binder selected from chicory root, a chicory root extract, inulin, or combinations thereof; and devoid or substantially devoid of any other binder, and wherein the composition is in a lozenge including a compressed, powdered lozenge form. Various embodiments of the disclosure further relate to methods of increasing absorption of one or more active ingredients of an oral formulation with a decreased excipient profile, comprising receiving an intraorally absorbable composition comprising one or more active ingredients; inserting the composition into the mouth of a subject; and allowing the composition to fully dissolve in the mouth of the subject, wherein the composition comprises a) 50-90% of one or more active ingredients; and b) 10-50% of an excipient blend comprising a lubricant; a binder selected from chicory root, a chicory root extract, inulin, or combinations thereof, and devoid or substantially devoid of any other binder, and wherein the composition is in a lozenge including a compressed, powdered lozenge form, and wherein the composition fully orally disintegrates within about 1-20 minutes upon oral administration to a subject. In various embodiments, the dietary fibers may include inulin, chicory root, and/or chicory root extracts. In various embodiments, suitable lubricants may include magnesium stearate, stearic acid or combinations thereof.

In various embodiments, the methods include increasing absorption of one or more active ingredients of an oral formulation of the compositions in powdered form. Preferentially, the methods include increasing absorption of one or more active ingredients of an oral formulation of powdered forms of the composition comprised of mixtures of active ingredients and an excipient blend that can be effectively compressed into solid oral dosage forms. Preferentially, the methods include increasing absorption of one or more active ingredients of an oral formulation of powdered forms of the composition comprised of mixtures of active ingredients and an excipient blend that can be effectively compressed into solid oral dosage forms that are lozenges including compressed, powdered lozenges. Preferentially, the methods include increasing absorption of one or more active ingredients of an oral formulation of powdered forms of the compositions comprised of mixtures of active ingredients and an excipient blend that can be effectively compressed into solid oral dosage forms that are lozenges including compressed, powdered lozenges that provide for efficient bioavailability of active ingredients by intraoral absorption.

In various embodiments, the methods include increasing absorption of one or more active ingredients of an oral formulation of compositions manufactured in solid oral dosage form. Preferentially, the methods include increasing absorption of one or more active ingredients of an oral formulation of solid oral dosage forms of the compositions comprised of mixtures of active ingredients and an excipient blend. Preferentially, the methods include increasing absorption of one or more active ingredients of an oral formulation of solid oral dosage forms of the compositions comprised of mixtures of active ingredients and an excipient blend that are lozenges including compressed, powdered lozenges. Preferentially, the methods include increasing absorption of one or more active ingredients of an oral formulation of solid oral dosage forms of the compositions comprised of mixtures of active ingredients and an excipient blend that are lozenges including compressed, powdered lozenges that provide for efficient bioavailability of active ingredients by intraoral absorption.

Preferentially, the methods include increasing absorption of one or more active ingredients of an oral formulation of the compositions that contain high amounts and high density of active ingredients.

Preferentially, the methods include increasing absorption of one or more active ingredients of an oral formulation of the compositions of that contain low amounts of excipient ingredients.

Preferentially, the methods include increasing absorption of one or more active ingredients of an oral formulation of the compositions that can be produced without the need for sugars, sugar derivatives, sugar substitutes, and sugar alcohols.

Preferentially, the methods include increasing absorption of one or more active ingredients of an oral formulation of the compositions that allow for high bioavailability after administration to a subject.

In various embodiments, the methods include increasing absorption of one or more active ingredients of an oral formulation of the compositions that may include an effective amount of active ingredients. Suitable active ingredients may include pharmaceutical agents, nutraceutical agents, and nutrients. In various embodiments, the methods include increasing absorption of one or more active ingredients of an oral formulation of the compositions that may include an effective amount of at least about 30 wt % of one or more active ingredients, or between about 30 wt % to about 95 wt %, or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 wt %, or any range between any two of these amounts including about 30 wt % to about 40 wt %, or about 30 wt % to about 50 wt %, or about 30 wt % to about 60 wt %, or about 30 wt % to about 70 wt %, or about 30 wt % to about 80 wt %, or about 30 wt % to about 90 wt %, or about 40 wt % to about 50 wt %, or about 40 wt % to about 60 wt %, or about 40 wt % to about 70 wt %, or about 40 wt % to about 80 wt %, or about 40 wt % to about 90 wt %, or about 50 wt % to about 60 wt %, or about 50 wt % to about 70 wt %, or about 50 wt % to about 80 wt %, or about 50 wt % to about 90 wt %, or about 60 wt % to about 70 wt %, or about 60 wt % to about 80 wt %, or about 60 wt % to about 90 wt %, or about 70 wt % to about 80 wt %, or about 70 wt % to about 90 wt %, or about 80 wt % to about 90 wt % of one or more active ingredients. In some preferred forms, the methods include increasing absorption of one or more active ingredients of an oral formulation wherein the amount of one or more active ingredients is sufficient to maintain an optimum level of one or more active ingredients in the systemic circulation of a subject receiving an administration of the composition on a daily basis.

In various embodiments, the methods include increasing absorption of one or more active ingredients of an oral formulation of the compositions that may include an amount of an excipient blend. Suitable excipient blends are comprised of one or more suitable binders and/or one or more suitable lubricants. In various embodiments, the methods include increasing absorption of one or more active ingredients of an oral formulation of the compositions that may include an amount of at least about 5 wt % of an excipient blend, or between about 5 wt % to about 70 wt %, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 5 wt % to about 10 wt %, or about 5 wt % to about 20 wt %, or about 5 wt % to about 30 wt %, or about 5 wt % to about 40 wt %, or about 5 wt % to about 50 wt %, or about 5 wt % to about 60 wt %, or about 5 wt % to about 70 wt %, or about 10 wt % to about 20 wt %, or about 10 wt % to about 30 wt %, or about 10 wt % to about 40 wt %, or about 10 wt % to about 50 wt %, or about 10 wt % to about 60 wt %, or about 10 wt % to about 70 wt %, or about 15 wt % to about 20 wt %, or about 15 wt % to about 30 wt %, or about 15 wt % to about 40 wt %, or about 15 wt % to about 50 wt %, or about 15 wt % to about 60 wt %, or about 15 wt % to about 70 wt %, or about 20 wt % to about 30 wt %, or about 20 wt % to about 40 wt %, or about 20 wt % to about 50 wt %, or about 20 wt % to about 60 wt %, or about 20 wt % to about 70 wt %, or about 30 wt % to about 40 wt %, or about 30 wt % to about 50 wt %, or about 30 wt % to about 60 wt %, or about 30 wt % to about 70 wt %, or about 40 wt % to about 50 wt %, or about 40 wt % to about 60 wt %, or about 40 wt % to about 70 wt %, or about 50 wt % to about 60 wt %, or about 50 wt % to about 70 wt %, or about 60 wt % to about 70 wt % of an excipient blend.

In various embodiments, the methods include increasing absorption of one or more active ingredients of an oral formulation of the compositions that may include an amount one or more binders. Suitable binders may include chicory root, a chicory root extract, inulin, or combinations thereof. In various embodiments, the methods include increasing absorption of one or more active ingredients of an oral formulation of the compositions that may include an amount of at least about 1 wt % of one or more binders, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more binders. Preferably, the methods include increasing absorption of one or more active ingredients of an oral formulation of the compositions that may include about 1 wt % to about 10 wt % of a binder selected from chicory root, a chicory root extract, inulin, or combinations thereof, and devoid or substantially devoid of any other binder.

In various embodiments, the methods include increasing absorption of one or more active ingredients of an oral formulation of the compositions that may be devoid of or substantially devoid of additional binders other than chicory root, a chicory root extract, inulin, or combinations thereof. The term “substantially devoid” refers to amounts that are less than 5 wt % of the composition, or between about 0 wt % to about 5 wt %, or about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt %, or any range between any two of these amounts including about 0 wt % to about 0.01 wt %, or about 0 wt % to about 0.05 wt %, or about 0 wt % to about 0.01 wt %, or about 0 wt % to about 1 wt %, or about 0 wt % to about 2.5 wt %, or about 0 wt % to about 5 wt %, or about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.1 wt % to about 1 wt %, or about 0.1 wt % to about 2.5 wt %, or about 0.1 wt % to about 5 wt %, or about 0.5 wt % to about 1 wt %, or about 0.5 wt % to about 2.5 wt %, or about 0.5 wt % to about 5 wt %, or about 1 wt % to about 2.5 wt %, or about 1 wt % to about 5 wt %, or about 2 wt % to about 5 wt %, or about 3 wt % to about 5 wt %, or about 4 wt % to about 5 wt % of the composition.

In various embodiments, the methods include increasing absorption of one or more active ingredients of an oral formulation of the compositions that may include an amount one or more lubricants. Suitable lubricants may include magnesium stearate, stearic acid or combinations thereof. In various embodiments, the methods include increasing absorption of one or more active ingredients of an oral formulation of the compositions that may include no lubricants. In various embodiments, the methods include increasing absorption of one or more active ingredients of an oral formulation of the compositions that may include an amount of at least about 0.5 wt % of one or more lubricants, or between about 0.5 wt % to about 30 wt %, or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 wt %, or any range between any two of these amounts including about 0.5 wt % to about 1 wt %, or about 0.5 wt % to about 2 wt %, or about 0.5 wt % to about 3 wt %, or about 0.5 wt % to about 4 wt %, or about 0.5 wt % to about 5 wt %, or about 0.5 wt % to about 10 wt %, or about 1 wt % to about 2 wt %, or about 1 wt % to about 3 wt %, or about 1 wt % to about 4 wt %, or about 1 wt % to about 5 wt %, or about 1 wt % to about 10 wt %, or about 1 wt % to about 15 wt %, or about 1 wt % to about 20 wt %, or about 1 wt % to about 25 wt %, or about 1 wt % to about 30 wt %, or about 2 wt % to about 5 wt %, or about 2 wt % to about 10 wt %, or about 2 wt % to about 15 wt %, or about 2 wt % to about 20 wt %, or about 2 wt % to about 25 wt %, or about 2 wt % to about 30 wt %, or about 3 wt % to about 5 wt %, or about 3 wt % to about 10 wt %, or about 3 wt % to about 15 wt %, or about 3 wt % to about 20 wt %, or about 3 wt % to about 25 wt %, or about 3 wt % to about 30 wt %, or about 4 wt % to about 5 wt %, or about 4 wt % to about 10 wt %, or about 4 wt % to about 15 wt %, or about 4 wt % to about 20 wt %, or about 4 wt % to about 25 wt %, or about 4 wt % to about 30 wt %, or about 5 wt % to about 10 wt %, or about 5 wt % to about 15 wt %, or about 5 wt % to about 20 wt %, or about 5 wt % to about 25 wt %, or about 5 wt % to about 30 wt %, or about 10 wt % to about 15 wt %, or about 10 wt % to about 20 wt %, or about 10 wt % to about 25 wt %, or about 10 wt % to about 30 wt %, or about 15 wt % to about 20 wt %, or about 15 wt % to about 25 wt %, or about 15 wt % to about 30 wt %, or about 20 wt % to about 25 wt %, or about 20 wt % to about 30 wt %, or about 25 wt % to about 30 wt % of one or more lubricants.

In various embodiments, the methods include increasing absorption of one or more active ingredients of an oral formulation of the compositions that may further include one or more flavoring agents. Suitable flavoring agents may include natural flavors, natural with other natural flavors, artificial flavors, and natural and artificial flavors. In various embodiments, the methods include increasing absorption of one or more active ingredients of an oral formulation of the compositions that may include no flavoring agents. In various embodiments, the methods include increasing absorption of one or more active ingredients of an oral formulation of the compositions that may include an amount of at least about 1 wt % of one or more flavoring agents, or between about 1 wt % to about 70 wt %, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %, or any range between any two of these amounts including about 1 wt % to about 5 wt %, or about 1 wt % to about 10 wt %, or about 1 wt % to about 20 wt %, or about 1 wt % to about 30 wt %, or about 1 wt % to about 40 wt %, or about 1 wt % to about 50 wt %, or about 1 wt % to about 60 wt %, or about 1 wt % to about 70 wt %, or about 2 wt % to about 10 wt %, or about 2 wt % to about 30 wt %, or about 2 wt % to about 30 wt %, or about 2 wt % to about 50 wt %, or about 2 wt % to about 70 wt %, or about 3 wt % to about 10 wt %, or about 3 wt % to about 20 wt %, or about 3 wt % to about 30 wt %, or about 3 wt % to about 50 wt %, or about 3 wt % to about 70 wt %, or about 4 wt % to about 10 wt %, or about 4 wt % to about 20 wt %, or about 4 wt % to about 30 wt %, or about 4 wt % to about 50 wt %, or about 4 wt % to about 70 wt %, or about 5 wt % to about 10 wt %, or about 4 wt % to about 20 wt %, or about 5 wt % to about 30 wt %, or about 5 wt % to about 50 wt %, or about 5 wt % to about 70 wt %, or about 10 wt % to about 20 wt %, or about 10 wt % to about 30 wt %, or about 10 wt % to about 50 wt %, or about 10 wt % to about 70 wt %, or about 20 wt % to about 30 wt %, or about 20 wt % to about 50 wt %, or about 20 wt % to about 70 wt %, or about 30 wt % to about 50 wt %, or about 30 wt % to about 70 wt %, or about 40 wt % to about 50 wt %, or about 40 wt % to about 70 wt %, or about 50 wt % to about 70 wt %, or about 60 wt % to about 70 wt % of one or more flavoring agents.

In various embodiments, the methods include increasing absorption of one or more active ingredients of an oral formulation of the compositions that may further include one or more sweeteners. Suitable sweeteners may include sugars, sugar derivatives, sugar substitutes, and sugar alcohols. In various embodiments, the methods include increasing absorption of one or more active ingredients of an oral formulation of the compositions that may include no sweeteners or substantially no sweeteners. In various embodiments, the methods include increasing absorption of one or more active ingredients of an oral formulation of the compositions that may include an amount of at least about 0.01 wt % of one or more sweeteners, or between about 0.01 wt % to about 10 wt %, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt %, or any range between any two of these amounts including about 0.01 wt % to about 0.05 wt %, or about 0.01 wt % to about 0.1 wt %, or about 0.01 wt % to about 1 wt %, or about 0.01 wt % to about 2.5 wt %, or about 0.01 wt % to about 5 wt %, or about 0.01 wt % to about 10 wt %, or about 0.03 wt % to about 0.05 wt %, or about 0.03 wt % to about 0.1 wt %, or about 0.03 wt % to about 1 wt %, or about 0.03 wt % to about 2.5 wt %, or about 0.03 wt % to about 5 wt %, or about 0.03 wt % to about 10 wt %, or about 0.05 wt % to about 0.1 wt %, or about 0.05 wt % to about 1 wt %, or about 0.05 wt % to about 2.5 wt %, or about 0.05 wt % to about 5 wt %, or about 0.05 wt % to about 10 wt %, or about 0.1 wt % to about 1 wt %, or about 0.1 wt % to about 2.5 wt %, or about 0.1 wt % to about 5 wt %, or about 0.1 wt % to about 10 wt %, or about 0.5 wt % to about 1 wt %, or about 0.5 wt % to about 2.5 wt %, or about 0.5 wt % to about 5 wt %, or about 0.5 wt % to about 10 wt %, or about 1 wt % to about 2.5 wt %, or about 1 wt % to about 5 wt %, or about 1 wt % to about 10 wt %, or about 2 wt % to about 5 wt %, or about 2 wt % to about 10 wt %, or about 3 wt % to about 5 wt %, or about 3 wt % to about 10 wt %, or about 4 wt % to about 10 wt %, or about 5 wt % to about 10 wt % of one or more sweeteners.

In various embodiments, the methods include increasing absorption of one or more active ingredients of an oral formulation of the compositions that orally disintegrate upon oral administration to a subject. In various embodiments, the methods include increasing absorption of one or more active ingredients of an oral formulation of the compositions of the disclosure that orally disintegrate in less than 30 minutes upon oral administration to a subject, or between about 1 min to about 30 min, or about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or 30 min, or any range between any two of these amounts including about 1 min to about 2 min, or about 1 min to about 3 min, or about 1 min to about 4 min, or about 1 min to about 5 min, or about 1 min to about 7.5 min, or about 1 min to about 10 min, or about 1 min to about 15 min, or about 1 min to about 20 min, or about 1 min to about 25 min, or about 1 min to about 30 min, or about 2 min to about 3 min, or about 2 min to about 4 min, or about 2 min to about 5 min, or about 2 min to about 7.5 min, or about 2 min to about 10 min, or about 2 min to about 15 min, or about 2 min to about 20 min, or about 2 min to about 25 min, or about 2 min to about 30 min, or about 3 min to about 4 min, or about 3 min to about 5 min, or about 3 min to about 7.5 min, or about 3 min to about 10 min, or about 3 min to about 15 min, or about 3 min to about 20 min, or about 3 min to about 25 min, or about 3 min to about 30 min, or about 4 min to about 5 min, or about 4 min to about 7.5 min, or about 4 min to about 10 min, or about 4 min to about 15 min, or about 4 min to about 20 min, or about 4 min to about 25 min, or about 4 min to about 30 min, or about 5 min to about 7.5 min, or about 5 min to about 10 min, or about 5 min to about 15 min, or about 5 min to about 20 min, or about 5 min to about 25 min, or about 5 min to about 30 min, or about 7.5 min to about 10 min, or about 7.5 min to about 15 min, or about 7.5 min to about 20 min, or about 7.5 min to about 25 min, or about 7.5 min to about 30 min, or about 10 min to about 15 min, or about 10 min to about 20 min, or about 10 min to about 25 min, or about 10 min to about 30 min, or about 15 min to about 20 min, or about 15 min to about 25 min, or about 15 min to about 30 min, or about 20 min to about 25 min, or about 20 min to about 30 min, or about 25 min to about 30 min upon oral administration to a subject.

### EXAMPLES

### Example 1—Example Amino Recovery Compositions

A) An example composition termed BCAA Blue Razz comprises the following ingredients:

| | |
|---|---|
| Amount | Ingredient |

| | | |
|---|---|---|
| 77.50 | kg | BCAA 2:1:1 (Instantized) |
| 31.00 | kg | L-Glutamine |
| 12.40 | kg | Malic Acid |
| 9.53 | kg | Citric Acid |
| 8.29 | kg | Blue Raspberry NAT (SP 100-104A) |
| 5.81 | kg | Sucralose |
| 4.11 | kg | Bitter Blocker NAT |
| 11.89 | kg | Chicory Root Fiber |
| 6.42 | kg | Magnesium Stearate |
| 166.95 | kg | Total |

Wherein BCAA 2:1:1 (Instantized) comprises a blend of the branched chain amino acids L-Leucine, L-Isoleucine, and L-Valine in a two-to-one-to-one ratio, respectively, and instantized similar to commercially available ingredients according to industry standards. In this example, active ingredients comprise about 108.5/166.95=65% and the excipient blend comprises about 35% including a binder that comprises about 11.89/166.95=7%, a lubricant that comprises about 6.42/166.95=4%, and flavor agents (including sweeteners) comprise 40.14/166.95=24% of the composition. Flavor agents plus the active ingredients equal 89%. A recommended serving of the composition is about 5.40 grams comprising about 4 compressed powdered lozenges of about 1.35 grams each.

B) An example composition termed BCAA Watermelon comprises the following ingredients:

| | |
|---|---|
| Amount | Ingredient |

| | | |
|---|---|---|
| 77.50 | kg | BCAA 2:1:1 (Instantized) |
| 31.00 | kg | L-Glutamine |
| 17.67 | kg | Malic Acid |
| 11.16 | kg | Citric Acid |
| 0.93 | kg | Beet Root Powder |
| 4.65 | kg | Watermelon N&A (SP 100-046A) |
| 3.10 | kg | Sucralose |
| 3.10 | kg | Silicon Dioxide |
| 2.02 | kg | Bitter Blocker NAT (SP 100-036K) |
| 12.40 | kg | Chicory Root Fiber |
| 6.54 | kg | Magnesium Stearate |
| 170.07 | kg | Total |

Wherein BCAA 2:1:1 (Instantized) comprises a blend of the branched chain amino acids L-Leucine, L-Isoleucine, and L-Valine in a two-to-one-to-one ratio, respectively, and instantized similar to commercially available ingredients according to industry standards. In this example, active ingredients comprise about 108.5/170.07=64% and the excipient blend comprises about 36% including a binder that comprises about 12.40/170.07=7%, a lubricant that comprises about 6.54/170.07=4% of the composition, and flavor agents (including sweeteners) comprise 38.6/170.07=23%, and a moisture absorber (silicon dioxide) that comprises 3.1/170.07=2%. Flavor agents plus the active ingredients equal 87%. A recommended serving of the composition is about 5.40 grams comprising about 4 compressed powdered lozenges of about 1.35 grams each.

### Example 2—Example Amino Energy Compositions

A) An example composition termed BCAA Energy Fruit Punch comprises the following ingredients:

| | |
|---|---|
| Amount | Ingredient |

| | | |
|---|---|---|
| 77.50 | kg | BCAA 2:1:1 (Instantized) |
| 31.00 | kg | Glutamine |
| 15.50 | kg | Beta Alanine |
| 3.10 | kg | Caffeine |
| 13.71 | kg | Malic Acid |
| 10.08 | kg | Citric Acid |
| 1.55 | kg | Beet Root Powder |
| 7.75 | kg | Fruit Punch NAT |
| 5.43 | kg | Sucralose |
| 4.65 | kg | Bitter Blocker NAT |
| 10.22 | kg | Chicory Root Fiber |
| 7.22 | kg | Magnesium Stearate |
| 187.71 | kg | Total |

Wherein BCAA 2:1:1 (Instantized) comprises a blend of the branched chain amino acids L-Leucine, L-Isoleucine, and L-Valine in a two-to-one-to-one ratio, respectively, and instantized similar to commercially available ingredients according to industry standards. In this example, active ingredients comprise about 124/187.71=66% and the excipient blend comprises about 34% including a binder that comprises about 10.22/187.71=5%, a lubricant that comprises about 7.22/187.71=4% of the composition, and flavor agents (including sweeteners) comprise 43.16/187.71=23%. Flavor agents plus the active ingredients equal 89%. A recommended serving of the composition is about 6.05 grams comprising about 4 compressed powdered lozenges of about 1.51 grams each.

B) An example composition termed BCAA Energy Orange Pineapple comprises the following ingredients:

| | |
|---|---|
| Amount | Ingredient |

| | | |
|---|---|---|
| 77.50 | kg | BCAA 2:1:1 (Instantized) |
| 31.00 | kg | Glutamine |
| 15.50 | kg | Beta Alanine |
| 3.10 | kg | Caffeine |
| 13.71 | kg | Malic Acid |
| 10.08 | kg | Citric Acid |
| 7.75 | kg | NAT Orange Pineapple Flavor |
| 5.43 | kg | Sucralose |
| 4.65 | kg | Bitter Blocker NAT |
| 10.12 | kg | Chicory Root Fiber |
| 7.15 | kg | Magnesium Stearate |
| 185.99 | kg | Total |

Wherein BCAA 2:1:1 (Instantized) comprises a blend of the branched chain amino acids L-Leucine, L-Isoleucine, and L-Valine in a two-to-one-to-one ratio, respectively, and instantized similar to commercially available ingredients according to industry standards. In this example, active ingredients comprise about 127.10/185.99=68% and the excipient blend comprises about 35.10/185.99=18.87% including a binder that comprises about 10.12/185.99=5%, a lubricant that comprises about 7.15/185.99=4%, and flavor agents (including sweeteners) comprise 41.61/185.99=22% of the composition. Flavor agents plus the active ingredients equal 90%. A recommended serving of the composition is about 6.00 grams comprising about 4 compressed powdered lozenges of about 1.50 grams each.

### Example 3—Example Pre-Workout Compositions

A) An example composition termed Pre-Workout Peach Mango comprises the following ingredients:

| | |
|---|---|
| Amount | Ingredient |

| | | |
|---|---|---|
| 42.00 | kg | Citrulline Malate 2:1 |
| 21.00 | kg | Beta Alanine |
| 3.15 | kg | Caffeine (Anhydrous) (Synthetic) |
| 4.20 | kg | Taurine |
| 21.00 | kg | Creatine Monohydrate |
| 3.45 | kg | Citric Acid |
| 1.26 | kg | Beta Carotene 1% |
| 3.68 | kg | Peach Mango NAT |
| 2.10 | kg | Sucralose |
| 1.84 | kg | Silicon Dioxide |
| 1.84 | kg | Calcium Silicate |
| 1.05 | kg | Bitter Blocker NAT |
| 4.26 | kg | Chicory Root Fiber |
| 4.43 | kg | Magnesium Stearate |
| 115.26 | kg | Total |

Wherein Citrulline Malate 2:1 comprises a blend of the α-amino acid citrulline and malic acid in a two-to-one ratio, respectively. In this example, active ingredients comprise about 91.35/115.26=79% and the excipient blend comprises about 21% including a binder that comprises about 4.26/115.26=4%, a lubricant that comprises about 4.43/115.26=4% of the composition, flavor agents (including sweeteners) comprise 11.53/166.95=10%, and moisture absorbers (silicon dioxide and calcium silicate) that comprise 3.68/166.95=3%. Flavor agents plus the active ingredients equal 89%. A recommended serving of the composition is about 5.69 grams comprising about 4 compressed powdered lozenges of about 1.42 grams each.

B) An example composition termed Pre-Workout Berry Lemonade comprises the following ingredients:

| | |
|---|---|
| Amount | Ingredient |

| | | |
|---|---|---|
| 42.00 | kg | Citrulline Malate 2:1 |
| 21.00 | kg | Creatine Monohydrate |
| 21.00 | kg | Beta Alanine |
| 3.15 | kg | Caffeine Anhydrous |
| 4.20 | kg | Taurine |
| 2.73 | kg | Citric Acid |
| 0.89 | kg | Beet Root Powder |
| 3.73 | kg | Lemon NAT WONF (SP 310-783A) |
| 2.10 | kg | Sucralose |
| 1.58 | kg | Silicon Dioxide |
| 1.58 | kg | Calcium Silicate |
| 1.05 | kg | Strawberry N&A (SP 300-676A) |
| 1.05 | kg | Bitter Blocker NAT (SP 100-036K) |
| 4.24 | kg | Chicory Root Fiber |
| 4.43 | kg | Magnesium Stearate |
| 114.73 | kg | Total |

Wherein Citrulline Malate 2:1 comprises a blend of the α-amino acid citrulline and malic acid in a two-to-one ratio, respectively. In this example, active ingredients comprise about 91.35/114.73=80% and the excipient blend comprises about 20% including a binder that comprises about 4.24/114.73=4%, a lubricant that comprises about 4.43/114.73=4% of the composition, flavor agents (including sweeteners) comprise 10.66/114.73=9%, and moisture absorbers that comprise 3.16/114.73=2%. Flavor agents plus the active ingredients equal about 89%. A recommended serving of the composition is about 5.69 grams comprising about 4 compressed powdered lozenges of about 1.42 grams each.

### Example 4—Example Electrolyte Energy Compositions

A) An example composition termed Electrolyte Lemon Lime comprises the following ingredients:

| | |
|---|---|
| Amount | Ingredient |

| | | |
|---|---|---|
| 84.00 | kg | Dextrose |
| 1.47 | kg | Potassium Citrate (36% Potassium) |
| 3.16 | kg | Himalayan Rock Salt (39% Sodium) |
| 10.32 | kg | Citric Acid |
| 4.20 | kg | Lemon NAT WONF |
| 1.01 | kg | Lime NAT |
| 1.68 | kg | Sucralose |
| 1.68 | kg | Bitter Blocker NAT |
| 4.30 | kg | Chicory Root Fiber |
| 4.47 | kg | Magnesium Stearate |
| 116.29 | kg | Total |

In this example, the active ingredients comprise about 88.63/116.29=76% and the excipient blend comprises about 24% including a binder that comprises about 4.30/116.29=4%, a lubricant that comprises about 4.47/116.29=4% of the composition, and flavor agents (including sweeteners) comprise about 18.89/116.29=16%. Flavor agents plus the active ingredients equal 92%. A recommended serving of the composition is about 5.54 grams comprising about 4 compressed powdered lozenges of about 1.39 grams each.

B) An example composition termed Electrolyte Grape comprises the following ingredients:

| | |
|---|---|
| Amount | Ingredient |

| | | |
|---|---|---|
| 84.00 | kg | Dextrose |
| 1.47 | kg | Potassium Citrate (36% Potassium) |
| 3.16 | kg | Himalayan Rock Salt (39% Sodium) |
| 11.33 | kg | Tartaric Acid |
| 5.88 | kg | Grape NAT (SP 300-735A) |
| 1.68 | kg | Sucralose |
| 1.68 | kg | Bitter Blocker NAT (SP 100-036K) |
| 4.37 | kg | Chicory Root Fiber |
| 4.54 | kg | Magnesium Stearate |
| 118.11 | kg | Total |

In this example, active ingredients comprise about 88.63/118.11=75% and the excipient blend comprises about 25% including a binder that comprises about 4.37/118.11=4%, a lubricant that comprises about 4.54/118.11=% of the composition, and flavor agents (including sweeteners) comprise 20.57/118.11=17%. Flavor agents plus the active ingredients equal 92%. A recommended serving of the composition is about 5.62 grams comprising about 4 compressed powdered lozenges of about 1.41 grams each.

The compressed, powdered lozenges from Examples 1-4 were made with the following process. Raw powder ingredients were loaded into a V-Type dry powder mixing and blending machine. These ingredients included all active ingredients, and flavoring components. Ingredients were added at their individual unique amounts per product blend not to exceed 100 kg of total product in the blender. Ingredients were blended at average time of 12-20 minutes. After active ingredients and flavoring reached a homogenous blend, chicory root powder was added at its individual amount unique to specific product blend for a secondary round of blending. Blending continued for 5-6 additional minutes. The third and final blend included the lubricant added at its individual amount. That third blend did not exceed 5-6 minutes.

After blending was complete, the final blend was immediately added to the hopper of a rotary tablet press. This multi-station rotary press included upper punches, lower punches, and middle die to catch, overfill and compress the powder blend in sizes equaling between 1 g-2 g total individual lozenge weight. Then the machine released final manufactured lozenges. This rotary press had between 17 to 34 individual stations and rotated at approximately 8-11 revolutions per minute, producing between 187-272 tablets per minute. Each blend produced approximately 67,000 lozenges per batch.

### Example 5—Example Absorption and Bioavailability Study

In this example, subjects were dosed either intraorally with a solid intraoral formulation or orally with a liquid oral formulation of an Electrolyte Fruit Punch composition including the following ingredients:

| | |
|---|---|
| Amount | Ingredient |

| | | |
|---|---|---|
| 4000.00 | mg | Dextrose |
| 70.00 | mg | Potassium Citrate (36% Potassium) |
| 150.38 | mg | Himalayan Rock Salt (39% Sodium) |
| 539.62 | mg | Tartaric Acid |
| 230.00 | mg | Fruit Punch NAT |
| 80.00 | mg | Sucralose |
| 80.00 | mg | Bitter Blocker NAT (SP 100-036K) |
| 208.00 | mg | Chicory Root Fiber |
| 216.32 | mg | Magnesium Stearate |
| 5574.32 | mg | Total |

Subjects were dosed with a single dose of about 5.62 grams of either formulation and blood glucose levels were monitored at baseline and at several points following baseline up to 50 minutes post dosing. Absorption and Bioavailability was recorded as the difference between the baseline blood glucose level and the blood glucose level at each time point for each cohort. Results displayed in FIGURES TA (bar chart) and 1B (line chart) suggest faster and higher overall absorption and bioavailability of the dextrose ingredient for the solid intraoral formulation cohort versus the liquid oral formulation cohort.

It will be apparent to those having skill in the art that the examples and embodiments disclosed are exemplary only and not intended to be limiting to the many variations of the details of the above-described examples and embodiments that are possible without departing from the underlying principles of the invention.

What is claimed is:

1. An intraorally absorbable composition, comprising
a. 50-90% of one or more active ingredients; and
b. 10-50% of an excipient blend comprising a lubricant; a binder selected from chicory root, a chicory root extract, inulin, or combinations thereof; and devoid or substantially devoid of any other binder,
wherein the composition is in a compressed, powdered lozenge form, and wherein the composition fully orally disintegrates within about 1-20 minutes upon oral administration to a subject.

2. The composition of any of claim 1, wherein the one or more active ingredients is present in an amount of at least 60 wt % of the composition.

3. The composition of claim 1, wherein the one or more active ingredients is present in an amount of at least 70 wt % of the composition.

4. The composition of claim 1, wherein the binder is present in an amount from about 1 wt % to about 10 wt % of the composition.

5. The composition of claim 1, wherein the binder is present in an amount from about 2 wt % to about 8 wt % of the composition.

6. The composition of claim 1, wherein the lubricant is present in an amount from about 0.5 wt % to about 5 wt % of the composition.

7. The composition of claim 1, wherein the lubricant is present in an amount from about 2 wt % to about 4 wt % of the composition.

8. The composition of claim 1, wherein the lubricant is magnesium stearate, stearic acid, or combinations thereof.

9. The composition of claim 1, wherein the excipient blend further comprises one or more flavoring agents.

10. The composition of claim 1, wherein the one or more active ingredients comprises one or more nutrients, nutraceutical agents, or combinations thereof.

11. The composition of claim 1, wherein the binder and the lubricant are present in a combined amount from about 2 wt % to about 15 wt % of the composition.

12. The composition of claim 1, wherein the binder and the lubricant are present in a combined amount from about 3 wt % to about 10 wt % of the composition.

13. The composition of claim 1, wherein the excipient blend further comprises one or more sweeteners, wherein the one or more sweeteners is present in an amount less than about 5 wt % of the composition, and wherein the one or more sweeteners is selected from the group consisting of sugars, sugar derivatives, sugar substitutes, sugar alcohols, and combinations thereof.

14. The composition of claim 13, in which the one or more sweeteners is substantially absent from the composition.

15. The composition of claim 1, wherein the composition fully orally disintegrates within about 10-20 minutes upon oral administration to a subject.

16. The composition of claim 1, wherein the composition fully orally disintegrates within about 2-9 minutes upon oral administration to a subject.

17. A method of making the intraorally absorbable composition of claim 1, comprising:
combining 50-90% of one or more active ingredients with 10-50% of an excipient blend comprising a lubricant; a binder selected from chicory root, a chicory root extract, inulin, or combinations thereof; and devoid or substantially devoid of any other binder; and
compressing the combination of the one or more active ingredients and the excipient blend at high pressure and low speed into a compressed, powdered lozenge form that fully orally disintegrates within about 1-20 minutes upon oral administration to a subject.

18. The method of claim 17, wherein the one or more active ingredients is present in an amount from about 60 wt % to about 90 wt % of the composition.

19. A method of increasing absorption of one or more active ingredients, the method comprising:
receiving the intraorally absorbable composition of claim 1 comprising the one or more active ingredients;
inserting the composition into the mouth of a subject; and
allowing the composition to fully dissolve in the mouth of the subject.

20. The method of claim 19, wherein the one or more active ingredients is present in an amount from about 60 wt % to about 90 wt % of the composition.
